(19) United States
(12) Patent Application Publication
(10) Pub. No.: US 2023/0097790 A1
(43) Pub. Date: Mar. 30, 2023

(54) SYSTEM AND METHOD FOR CAPTURING CARDIOPULMONARY SIGNALS

(71) Applicant: Apple Inc., Cupertino, CA (US)

(72) Inventors: Jun GONG, Austin, TX (US); Gierad LAPUT, Pittsburgh, PA (US); Seyedeh Fereshteh SHAHMIRI, Atlanta, GA (US)

(21) Appl. No.: 17/872,970

(22) Filed: Jul. 25, 2022

Related U.S. Application Data
(60) Provisional application No. 63/248,355, filed on Sep. 24, 2021.

(51) Int. Cl. A61B 7/00; A61B 5/08

(52) U.S. Cl. CPC A61B 7/003; A61B 5/08; A61B 5/0022

(57) ABSTRACT

A method is provided that includes receiving an accelerometer signal from an accelerometer in a headphone configured to be mounted in a user's ear canal and filtering the accelerometer signal to extract a cardiac signal. The method further includes detecting a plurality of peaks in the cardiac signal and determining a cardiac rate of the user based on the detected plurality of peaks.

### CROSS-REFERENCE TO RELATED APPLICATIONS

This application claims the benefit of U.S. Provisional Patent Application No. 63/248,355, entitled “SYSTEM AND METHOD FOR CAPTURING CARDIOPULMONARY SIGNALS”, filed Sep. 24, 2021, the entirety of which is incorporated herein by reference.

### TECHNICAL FIELD

The present description relates generally to capturing cardiopulmonary signals of a user including, for example, capturing cardiopulmonary signals using a high-speed accelerometer in headphones configured to be mounted in the user's ear canal.

### BACKGROUND

Physiological activities in the human body are main indicators of health factors. These activities yield a multitude of mechano-acoustic signatures that are a complex superposition of signals arising from body motions, respiration, cardiac activity, and other sources. Current systems for capturing aspects of these mechano-acoustic signals suffer from susceptibility to environmental noise or low-frequency motion artifacts, and have difficulty capturing both mechanical and acoustic signals simultaneously with high fidelity.

### BRIEF DESCRIPTION OF THE DRAWINGS

Certain features of the subject technology are set forth in the appended claims. However, for purpose of explanation, several embodiments of the subject technology are set forth in the following figures.

FIG. 1 illustrates an example network environment in which the subject technology may operate in accordance with one or more implementations.

FIG. 2 is a block diagram illustrating components of headphones and an electronic device in accordance with one or more implementations of the subject technology.

FIG. 3 is an example process for determining a cardiac rate from an accelerometer signal in one or more implementations of the subject technology.

FIG. 4 illustrates signals extracted from an accelerometer signal according to aspects of the subject technology.

FIG. 5 illustrates a peak detection process in accordance with one or more implementations of the subject technology.

FIG. 6 illustrates cardiopulmonary signals extracted from an accelerometer signal in accordance with one or more implementations of the subject technology.

FIG. 7 illustrates cardiopulmonary signals extracted from an accelerometer signal in accordance with one or more implementation of the subject technology.

FIG. 8 illustrates a peak detection process an example electronic system with which aspects of the subject technology may be implemented in accordance with one or more implementations.

### DETAILED DESCRIPTION

The detailed description set forth below is intended as a description of various configurations of the subject technology and is not intended to represent the only configurations in which the subject technology can be practiced. The appended drawings are incorporated herein and constitute a part of the detailed description. The detailed description includes specific details for the purpose of providing a thorough understanding of the subject technology. However, the subject technology is not limited to the specific details set forth herein and can be practiced using one or more other implementations. In one or more implementations, structures and components are shown in block diagram form in order to avoid obscuring the concepts of the subject technology.

The subject technology proposes the use of a high-speed accelerometer that combines the characteristics of an accelerometer and a contact microphone to capture the mechano-acoustic signals in multiple frequency bands while its signal is insusceptible to environmental noise. This coupling is helpful since it allows extracting cardiac features in multiple signals (e.g., ballistocardiogram (BCG) and phonocardiogram (PCG) signals), and therefore may be more effective in identifying cardiac activities.

According to aspects of the subject technology, high-speed accelerometers available in earbuds or headphones configured to mounted in the ear canal of a user may be used to capture the mechano-acoustic signals. These accelerometers benefit from a relatively stable position in the ear canal with respect to vital organs. Thus, robust measurements of cardiac and respiratory functions may be obtained from inside the ear. According to aspects of the subject technology, high-speed accelerometers having high sensitivity in a wide frequency spectrum with high dynamic range are used to capture the mechano-acoustic signals. For example, a high-speed accelerometer may have a 16 KHz sampling frequency and 24-bit resolution. However, the subject technology is not limited to these specifications and may be implemented with different sampling frequencies and sampling resolutions. For example, the sampling frequency may be as low as 2 KHz. Using these high-speed accelerometers allows a single sensor to simultaneously record multiple cardiopulmonary signals, from subtle vibrations produced by respiration and heart beats (e.g., BCG signals from DC to 20 Hz), to those acoustic waves produced by heart and lung sounds (e.g., PCG signals) that cover a wide spectrum ranging over 1 Hz or less, to over 2 KHz. In addition, cardiopulmonary signals may be acquired without user intervention, longitudinally and in different physical activity conditions. For purposes of this description, the term “accelerometer” will reference the high-speed accelerometer described above.

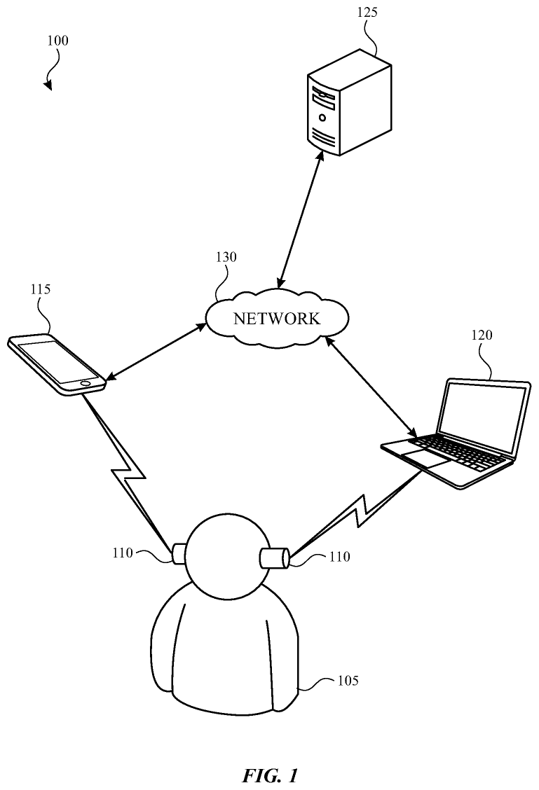

FIG. 1 illustrates an example network environment 100 in which the subject technology may operate in accordance with one or more implementations. Not all of the depicted components may be used in all implementations, however, and one or more implementations may include additional or different components than those shown in the figure. Variations in the arrangement and type of the components may be made without departing from the spirit or scope of the claims as set forth herein. Additional components, different components, or fewer components may be provided.

As illustrated in FIG. 1, network environment 100 includes user 105 wearing headphones 110, electronic devices 115 and 120, server 125, and network 130. Headphones 110 represent earbuds or headphones configured to be mounted in the ear-canals of user 105. Headphones 110 may be communicatively coupled to either electronic device 115 and/or electronic device 120 via a wireless or wired connection. For example, headphones 110 may be communicatively coupled to electronic device 115 using a Bluetooth connection.

Network 130 may communicatively (directly or indirectly) couple electronic devices 115 and 120 in a local network environment. Additionally, LAN 140 may communicatively couple (directly or indirectly) electronic devices 115 and 120 to server 125. In one or more implementations, network 130 may include one or more different network devices/network medium and/or may utilize one or more different wireless and/or wired network technologies, such as Ethernet, optical, Wi-Fi, Bluetooth, Zigbee, Powerline over Ethernet, coaxial, Ethernet, Z-Wave, cellular, or generally any wireless and/or wired network technology that may communicatively couple two or more devices. In one or more implementations, network 130 may be an interconnected network of devices that may include, and/or may be communicatively coupled to, the Internet. For explanatory purposes, network environment 100 is illustrated in FIG. 1 as including electronic devices 115 and 120, and server 125; however, network environment 100 may include any number of electronic devices and any number of servers.

FIG. 1 illustrates electronic device 115 as a smartphone and electronic device 120 as a laptop computer. The subject technology is not limited to these types or numbers of electronic devices. For example, any of electronic devices 115 and 120 may be a portable computing device such as a laptop computer, a smartphone, a set top box including a digital media player, a tablet device, a wearable device such as a smartwatch or a band, or any other appropriate device that is capable of executing client applications, providing access to the client applications via a graphical user interface, and includes and/or is communicatively coupled to, for example, one or more wired or wireless interfaces, such as WLAN radios, cellular radios, Bluetooth radios, Zigbee radios, near field communication (NFC) radios, and/or other wireless radios.

Server 125 represents one or more computing devices that are configured to provide services to users via client applications being executed on electronic devices 115 and/or 120. For example, server 125 may provide healthcare or telemedicine services with which cardiopulmonary signals may be shared with a doctor or other healthcare professional. The subject technology is not limited to this number of services or these types of services.

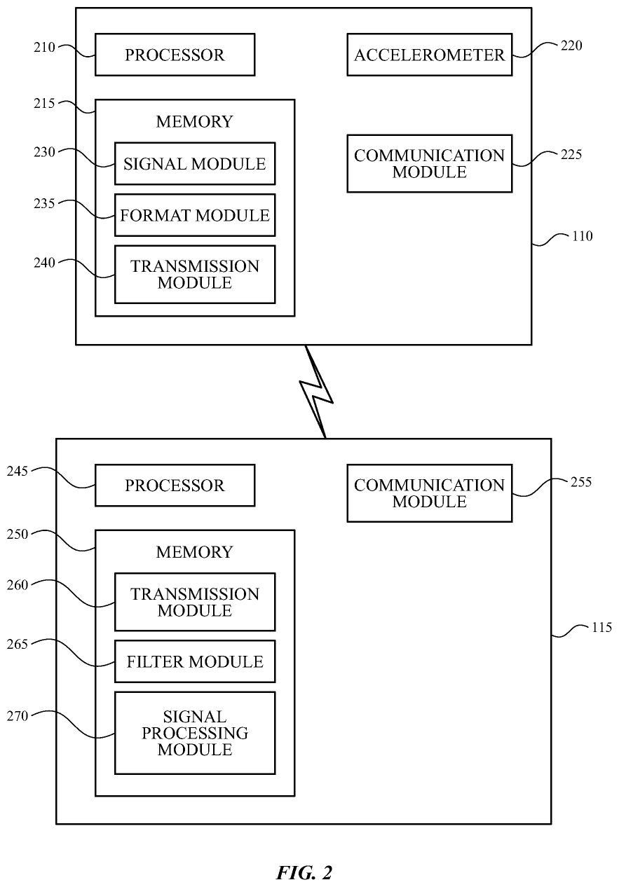

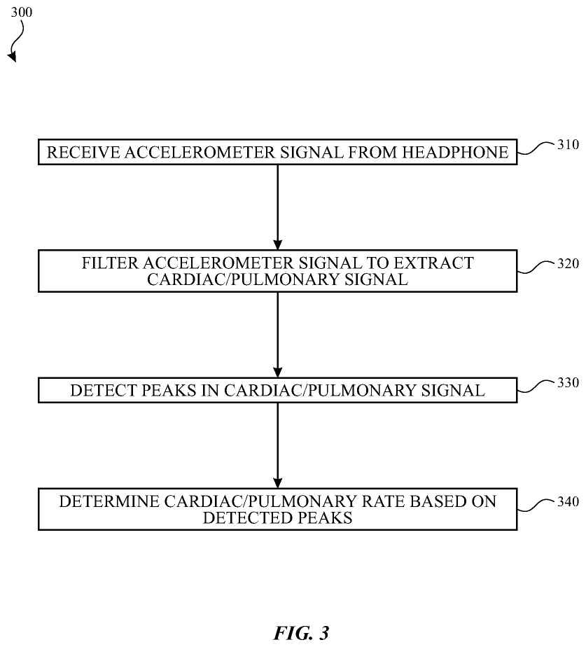

FIG. 2 is a block diagram illustrating components of headphones 110 and electronic device 115 in accordance with one or more implementations of the subject technology. While FIG. 2 depicts components for electronic device 115, FIG. 2 can correspond to electronic device 120 in FIG. 1 as well. Not all of the depicted components may be used in all implementations, however, and one or more implementations may include additional or different components than those shown in the figure. Variations in the arrangement and type of the components may be made without departing from the spirit or scope of the claims as set forth herein. Additional components, different components, or fewer components may be provided.

In the example depicted in FIG. 2, headphones 110 includes processor 210, memory 215, accelerometer 220, and communication module 225. Processor 210 may include suitable logic, circuitry, and/or code that enable processing data and/or controlling operations of headphones 110. In this regard, processor 210 may be enabled to provide control signals to various other components of headphones 110. Processor 210 may also control transfers of data between various portions of headphones 110. Additionally, the processor 210 may enable implementation of an operating system or otherwise execute code to manage operations of headphones 110.

Processor 210 or one or more portions thereof, may be implemented in software (e.g., instructions, subroutines, code), may be implemented in hardware (e.g., an Application Specific Integrated Circuit (ASIC), a Field Programmable Gate Array (FPGA), a Programmable Logic Device (PLD), a controller, a state machine, gated logic, discrete hardware components, or any other suitable devices) and/or a combination of both.

Memory 215 may include suitable logic, circuitry, and/or code that enable storage of various types of information such as received data, generated data, code, and/or configuration information. Memory 215 may include, for example, random access memory (RAM), read-only memory (ROM), flash memory, and/or magnetic storage. As depicted in FIG. 2, memory 215 contains signal module 230, format module 235, and transmission module 240. The subject technology is not limited to these components both in number and type, and may be implemented using more components or fewer components than are depicted in FIG. 2.

According to aspects of the subject technology, signal module 230 comprises a computer program having one or more sequences of instructions or code together with associated data and settings. Upon executing the instructions or code, one or more processes are initiated to capture cardiac or cardiopulmonary signals using accelerometer 220. Accelerometer 220 may be configured to capture the signals at a sampling frequency of at least 2 kHz (e.g., 16 kHz). Signal module 230 may be configured to read out the measurements made by accelerometer 220 at the sampling frequency to detect the cardiac or cardiopulmonary signals.

According to aspects of the subject technology, format module 235 comprises a computer program having one or more sequences of instructions or code together with associated data and settings. Upon executing the instructions or code, one or more processes are initiated to format the cardiac or cardiopulmonary signals into an audio format. For example, the signals may be formatted into a linear pulse code modulated format. Formatting the signals into an audio format allows headphones 115 to take advantage of existing communications systems for communicating signals with electronic device 115.

According to aspects of the subject technology, transmission module 240 comprises a computer program having one or more sequences of instructions or code together with associated data and settings. Upon executing the instructions or code, one or more processes are initiated to transmit the formatted cardiac or cardiopulmonary signals to electronic device 115 via communication module 225. Communication module 225 represents the interface for wireless or wired communications with electronic device 115. For example, communications module 225 may implement the Bluetooth standard for wireless communications between devices.

In the example depicted in FIG. 2, electronic device 115 includes processor 245, memory 250, accelerometer 220, and communication module 255. Processor 245 may include suitable logic, circuitry, and/or code that enable processing data and/or controlling operations of electronic device 115. In this regard, processor 245 may be enabled to provide control signals to various other components of electronic device 115. Processor 245 may also control transfers of data between various portions of electronic device 115. Additionally, the processor 245 may enable implementation of an operating system or otherwise execute code to manage operations of electronic device 115.

Processor 245 or one or more portions thereof, may be implemented in software (e.g., instructions, subroutines, code), may be implemented in hardware (e.g., an Application Specific Integrated Circuit (ASIC), a Field Programmable Gate Array (FPGA), a Programmable Logic Device (PLD), a controller, a state machine, gated logic, discrete hardware components, or any other suitable devices) and/or a combination of both.

Memory 250 may include suitable logic, circuitry, and/or code that enable storage of various types of information such as received data, generated data, code, and/or configuration information. Memory 250 may include, for example, random access memory (RAM), read-only memory (ROM), flash memory, and/or magnetic storage. As depicted in FIG. 2, memory 250 contains transmission module 260, filter module 265, and signal processing module 270. The subject technology is not limited to these components both in number and type, and may be implemented using more components or fewer components than are depicted in FIG. 2.

According to aspects of the subject technology, transmission module 260 comprises a computer program having one or more sequences of instructions or code together with associated data and settings. Upon executing the instructions or code, one or more processes are initiated to receive the formatted cardiac or cardiopulmonary signals from headphones 110 via communication module 255. Communication module 255 represents the interface for wireless or wired communications with headphones 110. For example, communications module 255 may implement the Bluetooth standard for wireless communications between devices.

According to aspects of the subject technology, filter module 265 comprises a computer program having one or more sequences of instructions or code together with associated data and settings. Upon executing the instructions or code, one or more processes are initiated to filter the cardiac or cardiopulmonary signal received from an accelerometer of headphones 110 to extract specific cardiac and pulmonary signals. The signal received from headphones 110 is a superposition signal consisting of mechanical vibrations produced by the user's cardiac and respiratory systems, body motions, and other potentially physiological vibrations produced by the body and propagated inside the ear canal. In addition, extra noises such as motion artifacts produced by body and/or device movements are inherent in the superposition signal. According to aspects of the subject technology, the signal received from headphones 110 is filtered to extract the signal(s) of interest. For example, one or more band-pass filters, such as a finite impulse response Butterworth band-pass filter, may be used to extract the signals of interest. BCG and SCG signals may be extracted in the 0.01 to 25 Hz frequency range, PCG signals may be extracted in the 25 to 240 Hz frequency range, and lung sounds or respiratory signals may be extracted in the 80 to 500 Hz frequency range. The subject technology is not limited to these specific ranges and signals of interest may be extracted using different sub-ranges or ranges than those listed above.

According to aspects of the subject technology, signal process module 270 comprises a computer program having one or more sequences of instructions or code together with associated data and settings. Upon executing the instructions or code, one or more processes are initiated to process the extracted signals to determine a cardiac or cardiopulmonary rate. Using an extracted PCG signal as an example, a process for detecting peaks and determining a cardiac rate will now be described. According to aspects of the subject technology, a gradient-based algorithm may be used to detect peaks that correspond to S1 and S2 sounds in the PCG signal. The algorithm first normalizes the signal. The algorithm then searches for the local maxima via a moving window in the time series. The time duration of the moving window may be 150 milliseconds, which approximately corresponds to the maximal length of a heart sound. The global maximum within the 150 milliseconds moving window is considered a potential heart sound component, either S1 or S2 sound. To further remove the false detected peaks, the algorithm checks the time interval between consecutive peaks, and ignores the one with intervals shorter than 0.33 seconds (˜180 bpm) and longer than 1.2 s (˜50 bpm). Applying a 6 second, 75% overlapping moving average window to peak-to-peak intervals yields an estimate of cardiac rate. The subject technology is not limited to the specific example described above and may be implemented with different parameters for the algorithm.

The foregoing process for peak detection and cardiac rate determination was described in the context of processing a PCG signal. Low frequency signals like BCG, however, can be difficult to identify peaks as the signals may get combined with motion artifacts. For signals like BCG, template matching may be used to detect the peaks in the signal.

FIG. 3 illustrates an example process for determining a cardiac rate from an accelerometer signal according to aspects of the subject technology. For explanatory purposes, the blocks of the process 300 are described herein as occurring in serial, or linearly. However, multiple blocks of the process 300 may occur in parallel. In addition, the blocks of the process 300 need not be performed in the order shown and/or one or more blocks of the process 300 need not be performed and/or can be replaced by other operations.

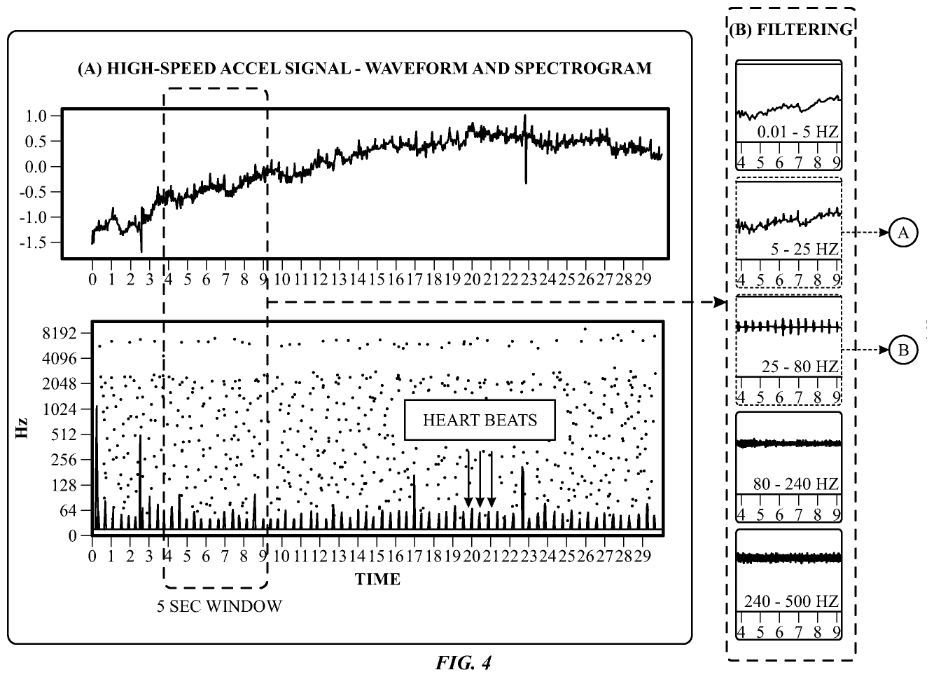

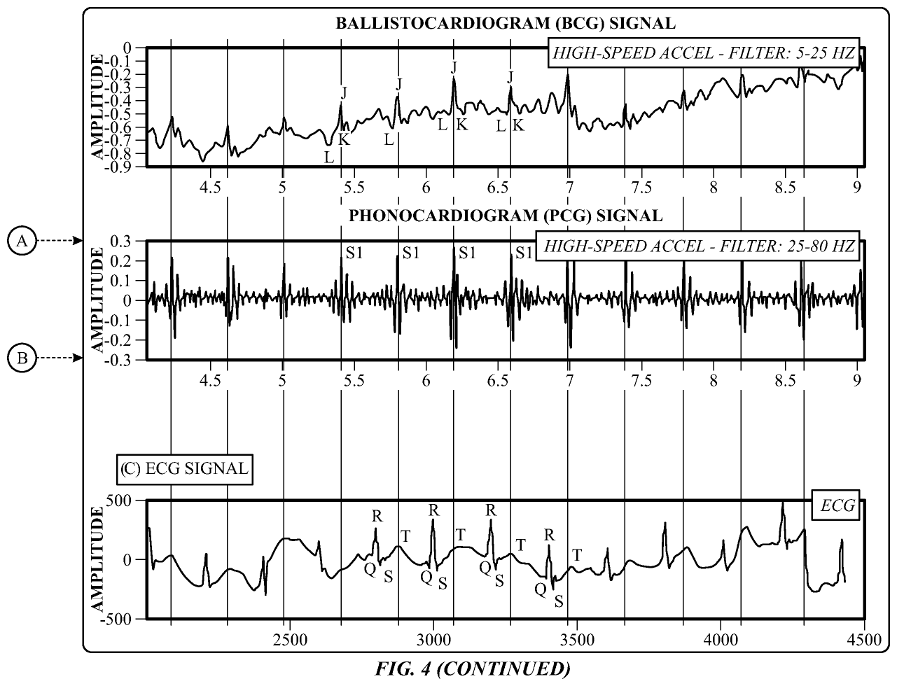

Example process 300 may be initiated upon electronic device 115 receiving an accelerometer signal from headphones 110 (block 310). As noted above, the accelerometer signal received from headphones 110 is a superposition signal from which cardiac signals of interest are extracted by filtering the accelerometer signal (block 320). FIG. 4 depicts representations of the accelerometer signal (A) along with the extracted signals (B) that were extracted using a 0.01-5 Hz bandpass filter, a 5-25 Hz bandpass filter, a 25-80 Hz bandpass filter, an 80-240 Hz bandpass filter, and a 240-500 Hz bandpass filter. Also shown for reference is an ECG signal (C) captured using another electronic device, such as a smartwatch worn on a user's wrist, for comparison with the other signals.

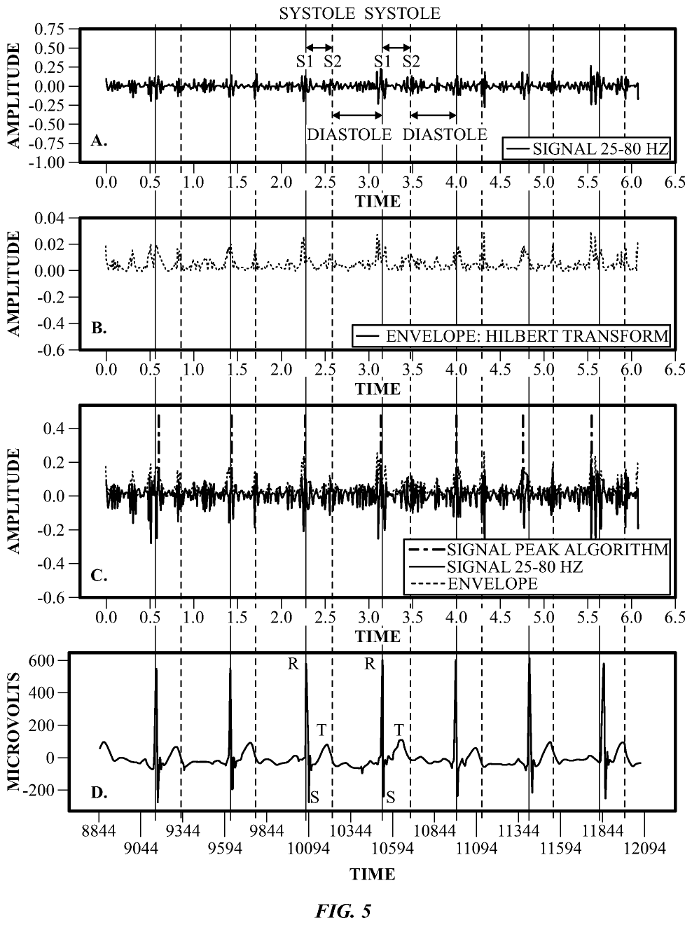

Using one of the extracted cardiac signals, such as a PCG signal, peaks are detected in the extracted signal using one of the processes described above (block 330). FIG. 5 depicts representations of peak detection process according to aspects of the subject technology. FIG. 5 includes a PCG cardiac signal (a.) extracted from an accelerometer signal using a bandpass filter with a frequency range of 25-80 Hz. As discussed above, an envelope of the PCG cardiac signal (b.) is extracted and peaks are detected (c.). Also shown for reference is an ECG signal (d.) captured using another electronic device, such as a smartwatch worn on a user's wrist.

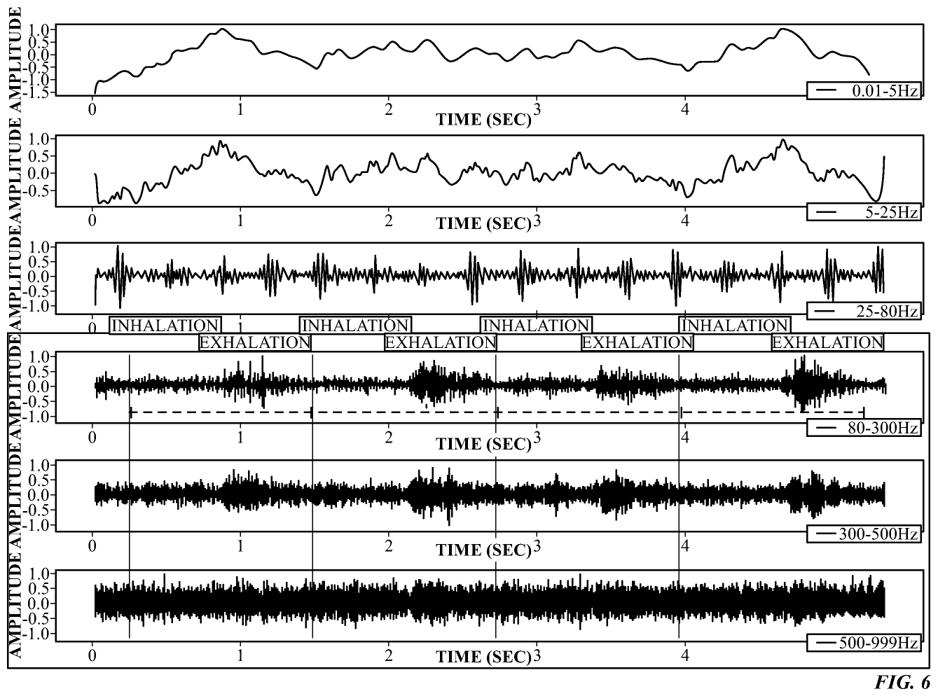

FIG. 6 depicts representations of signals extracted from a received accelerometer signal according to aspects of the subject technology. FIG. 6 includes signals extracted using 0.01-5 Hz bandpass filter, 5-25 Hz bandpass filter, 25-80 Hz bandpass filter, 80-300 Hz bandpass filter, and 500-999 Hz bandpass filter. As evidenced by the extracted signals, pulmonary signals can be extracted in the 80-300 Hz frequency range and the 300-500 Hz frequency range. From these signals, pulmonary or respiratory rates may be determined using peak detection and a moving average window as described above with respect to the PCG signal.

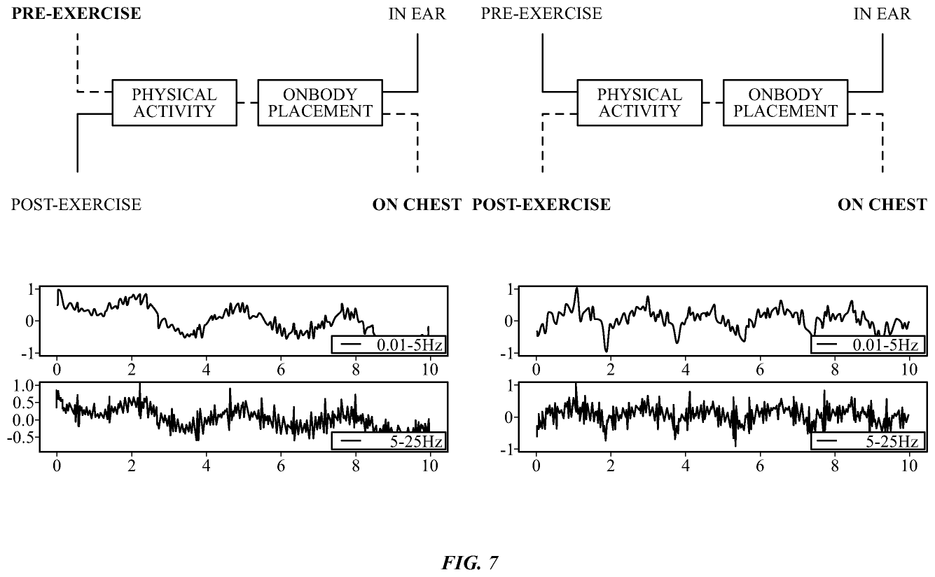

As described above, the accelerometer signal being processed may be obtained while headphones are mounted in a user's ear canal. According to aspects of the subject technology, cardiopulmonary signals may be extracted from an accelerometer signal obtained by placing one of the headphones on a cardiopulmonary landmark on the user's body, such as the user's chest. FIG. 4 illustrates an accelerometer signal obtained while headphones are mounted in the user's ear canal. FIGS. 5, 6, and 7 depict representations of cardiopulmonary signals extracted from an accelerometer signal obtained by placing the portion of a headphone containing the accelerometer on the user's chest before and after exercising. As evidenced by the depicted signals in FIG. 7, pulmonary or respiratory rates may be determined using peak detection and moving average window for both the signal extracted using the 0.01-5 Hz bandpass filter and the 5-25 Hz bandpass filter. The subject technology is not limited to the chest as the only cardiopulmonary landmark to obtain an accelerometer signal for processing the in the manner described above.

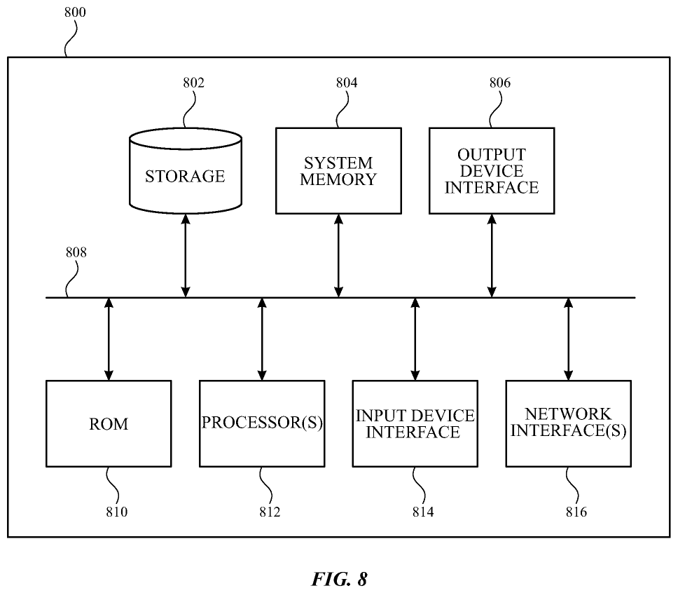

FIG. 8 illustrates an electronic system 800 with which one or more implementations of the subject technology may be implemented. Electronic system 800 can be, and/or can be a part of, one or more of electronic devices 115 and 120, or server 125 shown in FIG. 1. The electronic system 800 may include various types of computer readable media and interfaces for various other types of computer readable media. The electronic system 800 includes a bus 808, one or more processing unit(s) 812, a system memory 804 (and/or buffer), a ROM 810, a permanent storage device 802, an input device interface 814, an output device interface 806, and one or more network interfaces 816, or subsets and variations thereof.

The bus 808 collectively represents all system, peripheral, and chipset buses that communicatively connect the numerous internal devices of the electronic system 800. In one or more implementations, the bus 808 communicatively connects the one or more processing unit(s) 812 with the ROM 810, the system memory 804, and the permanent storage device 802. From these various memory units, the one or more processing unit(s) 812 retrieves instructions to execute and data to process in order to execute the processes of the subject disclosure. The one or more processing unit(s) 812 can be a single processor or a multi-core processor in different implementations.

The ROM 810 stores static data and instructions that are needed by the one or more processing unit(s) 812 and other modules of the electronic system 800. The permanent storage device 802, on the other hand, may be a read-and-write memory device. The permanent storage device 802 may be a non-volatile memory unit that stores instructions and data even when the electronic system 800 is off. In one or more implementations, a mass-storage device (such as a magnetic or optical disk and its corresponding disk drive) may be used as the permanent storage device 802.

In one or more implementations, a removable storage device (such as a floppy disk, flash drive, and its corresponding disk drive) may be used as the permanent storage device 802. Like the permanent storage device 802, the system memory 804 may be a read-and-write memory device. However, unlike the permanent storage device 802, the system memory 804 may be a volatile read-and-write memory, such as random access memory. The system memory 804 may store any of the instructions and data that one or more processing unit(s) 812 may need at runtime. In one or more implementations, the processes of the subject disclosure are stored in the system memory 804, the permanent storage device 802, and/or the ROM 810. From these various memory units, the one or more processing unit(s) 812 retrieves instructions to execute and data to process in order to execute the processes of one or more implementations.

The bus 808 also connects to the input and output device interfaces 814 and 806. The input device interface 814 enables a user to communicate information and select commands to the electronic system 800. Input devices that may be used with the input device interface 814 may include, for example, alphanumeric keyboards and pointing devices (also called “cursor control devices”). The output device interface 806 may enable, for example, the display of images generated by electronic system 800. Output devices that may be used with the output device interface 806 may include, for example, printers and display devices, such as a liquid crystal display (LCD), a light emitting diode (LED) display, an organic light emitting diode (OLED) display, a flexible display, a flat panel display, a solid state display, a projector, or any other device for outputting information. One or more implementations may include devices that function as both input and output devices, such as a touchscreen. In these implementations, feedback provided to the user can be any form of sensory feedback, such as visual feedback, auditory feedback, or tactile feedback; and input from the user can be received in any form, including acoustic, speech, or tactile input.

Finally, as shown in FIG. 8, the bus 808 also couples the electronic system 800 to one or more networks and/or to one or more network nodes through the one or more network interface(s) 816. In this manner, the electronic system 800 can be a part of a network of computers (such as a LAN, a wide area network (“WAN”), or an Intranet, or a network of networks, such as the Internet. Any or all components of the electronic system 800 can be used in conjunction with the subject disclosure.

Implementations within the scope of the present disclosure can be partially or entirely realized using a tangible computer-readable storage medium (or multiple tangible computer-readable storage media of one or more types) encoding one or more instructions. The tangible computer-readable storage medium also can be non-transitory in nature.

The computer-readable storage medium can be any storage medium that can be read, written, or otherwise accessed by a general purpose or special purpose computing device, including any processing electronics and/or processing circuitry capable of executing instructions. For example, without limitation, the computer-readable medium can include any volatile semiconductor memory, such as RAM, DRAM, SRAM, T-RAM, Z-RAM, and TTRAM. The computer-readable medium also can include any non-volatile semiconductor memory, such as ROM, PROM, EPROM, EEPROM, NVRAM, flash, nvSRAM, FeRAM, FeTRAM, MRAM, PRAM, CBRAM, SONOS, RRAM, NRAM, racetrack memory, FJG, and Millipede memory.

Further, the computer-readable storage medium can include any non-semiconductor memory, such as optical disk storage, magnetic disk storage, magnetic tape, other magnetic storage devices, or any other medium capable of storing one or more instructions. In one or more implementations, the tangible computer-readable storage medium can be directly coupled to a computing device, while in other implementations, the tangible computer-readable storage medium can be indirectly coupled to a computing device, e.g., via one or more wired connections, one or more wireless connections, or any combination thereof.

Instructions can be directly executable or can be used to develop executable instructions. For example, instructions can be realized as executable or non-executable machine code or as instructions in a high-level language that can be compiled to produce executable or non-executable machine code. Further, instructions also can be realized as or can include data. Computer-executable instructions also can be organized in any format, including routines, subroutines, programs, data structures, objects, modules, applications, applets, functions, etc. As recognized by those of skill in the art, details including, but not limited to, the number, structure, sequence, and organization of instructions can vary significantly without varying the underlying logic, function, processing, and output.

While the above discussion primarily refers to microprocessor or multi-core processors that execute software, one or more implementations are performed by one or more integrated circuits, such as ASICs or FPGAs. In one or more implementations, such integrated circuits execute instructions that are stored on the circuit itself.

Those of skill in the art would appreciate that the various illustrative blocks, modules, elements, components, methods, and algorithms described herein may be implemented as electronic hardware, computer software, or combinations of both. To illustrate this interchangeability of hardware and software, various illustrative blocks, modules, elements, components, methods, and algorithms have been described above generally in terms of their functionality. Whether such functionality is implemented as hardware or software depends upon the particular application and design constraints imposed on the overall system. Skilled artisans may implement the described functionality in varying ways for each particular application. Various components and blocks may be arranged differently (e.g., arranged in a different order, or partitioned in a different way) all without departing from the scope of the subject technology.

In accordance with the subject disclosure, a method is provided that includes receiving an accelerometer signal from an accelerometer in a headphone configured to be mounted in a user's ear canal and filtering the accelerometer signal to extract a cardiac signal. The method further includes detecting a plurality of peaks in the cardiac signal and determining a cardiac rate of the user based on the detected plurality of peaks.

The accelerometer signal may be parsed into an audio format. The audio format is may be a linear pulse code modulated format. Filtering the accelerometer signal may include applying a bandpass filter to the accelerometer signal. The bandpass filter may be a finite impulse response Butterworth bandpass filter. Detecting the plurality of peaks in the cardiac signal may include performing template matching on the cardiac signal. The method may further include extracting an envelope of the cardiac signal, where the plurality of peaks are detected in the extracted envelope of the cardiac signal.

In accordance with the subject disclosure, a non-transitory computer-readable medium storing instructions which, when executed by one or more processors, cause the one or more processors to perform operations is provided. The operations include receiving an accelerometer signal from an accelerometer in a headphone configured to be mounted in a user's ear canal and filtering the accelerometer signal to extract a first cardiac signal and a second cardiac signal. The operations further include detecting a first plurality of peaks in the first cardiac signal and a second plurality of peaks in the second cardiac signal and determining a first cardiac rate of the user based on the detected first plurality of peaks and a second cardiac rate of the user based on the detected second plurality of peaks.

The accelerometer signal may be parsed into an audio format. The audio format may be a linear pulse code modulated format. Filtering the accelerometer signal may include applying a first bandpass filter to the accelerometer signal to extract the first cardiac signal and a second bandpass filter to the accelerometer signal to extract the second cardiac signal. Detecting the plurality of peaks in the cardiac signal may include performing template matching on the first cardiac signal. The operations may further include extracting an envelope of the second cardiac signal, where the plurality of peaks are detected in the extracted envelope of the second cardiac signal.

In accordance with the subject disclosure, an electronic device is provided that includes a memory storing one or more computer programs and one or more processors configured to execute instructions of the one or more computer programs. Upon executing the instructions, the one or more processors receive an accelerometer signal from an accelerometer in a headphone configured to be mounted in a user's ear canal and filter the accelerometer signal to extract a cardiopulmonary signal. The one or more processors further detect a plurality of peaks in the cardiopulmonary signal and determine a cardiopulmonary rate of the user based on the detected plurality of peaks.

The accelerometer signal may be parsed into an audio format. Filtering the accelerometer signal may include applying a bandpass filter to the accelerometer signal. The cardiopulmonary rate of the user may be a respiratory rate of the user. The headphone may be positioned on the chest of the user.

In accordance with the subject disclosure, a headphone is provided that includes an accelerometer, a communication module, a memory storing one or more computer programs, and a processor configured to execute instructions in the one or more computer programs. Upon executing the instructions, the processor captures an accelerometer signal from the accelerometer, parses the accelerometer signal into an audio format, and transmits the accelerometer signal in the audio format to an electronic device using the communication module.

The audio format may be a linear pulse code modulated format. The communication module may be a wireless communication module.

As described herein, aspects of the subject technology may include the collection and transfer of data from an application to other computing devices. The present disclosure contemplates that in some instances, this collected data may include personal information data that uniquely identifies or can be used to identify a specific person. Such personal information data can include demographic data, location-based data, online identifiers, telephone numbers, email addresses, home addresses, images, data or records relating to a user's health or level of fitness (e.g., vital signs measurements, medication information, exercise information), date of birth, or any other personal information.

The present disclosure recognizes that the use of such personal information data, in the present technology, can be used to the benefit of users. For example, the personal information data can be used in syndicating content. Further, other uses for personal information data that benefit the user are also contemplated by the present disclosure. For instance, health and fitness data may be used, in accordance with the user's preferences to provide insights into their general wellness, or may be used as positive feedback to individuals using technology to pursue wellness goals.

The present disclosure contemplates that those entities responsible for the collection, analysis, disclosure, transfer, storage, or other use of such personal information data will comply with well-established privacy policies and/or privacy practices. In particular, such entities would be expected to implement and consistently apply privacy practices that are generally recognized as meeting or exceeding industry or governmental requirements for maintaining the privacy of users. Such information regarding the use of personal data should be prominently and easily accessible by users, and should be updated as the collection and/or use of data changes. Personal information from users should be collected for legitimate uses only. Further, such collection/sharing should occur only after receiving the consent of the users or other legitimate basis specified in applicable law. Additionally, such entities should consider taking any needed steps for safeguarding and securing access to such personal information data and ensuring that others with access to the personal information data adhere to their privacy policies and procedures. Further, such entities can subject themselves to evaluation by third parties to certify their adherence to widely accepted privacy policies and practices. In addition, policies and practices should be adapted for the particular types of personal information data being collected and/or accessed and adapted to applicable laws and standards, including jurisdiction-specific considerations which may serve to impose a higher standard. For instance, in the US, collection of or access to certain health data may be governed by federal and/or state laws, such as the Health Insurance Portability and Accountability Act (HIPAA); whereas health data in other countries may be subject to other regulations and policies and should be handled accordingly.

Despite the foregoing, the present disclosure also contemplates implementations in which users selectively block the use of, or access to, personal information data. That is, the present disclosure contemplates that hardware and/or software elements can be provided to prevent or block access to such personal information data. For example, in the case of video conferencing, the present technology can be configured to allow users to select to “opt in” or “opt out” of participation in the collection of personal information data during registration for services or anytime thereafter. In addition to providing “opt in” and “opt out” options, the present disclosure contemplates providing notifications relating to the access or use of personal information. For instance, a user may be notified upon downloading an app that their personal information data will be accessed and then reminded again just before personal information data is accessed by the app.

Moreover, it is the intent of the present disclosure that personal information data should be managed and handled in a way to minimize risks of unintentional or unauthorized access or use. Risk can be minimized by limiting the collection of data and deleting data once it is no longer needed. In addition, and when applicable, including in certain health related applications, data de-identification can be used to protect a user's privacy. De-identification may be facilitated, when appropriate, by removing identifiers, controlling the amount or specificity of data stored (e.g., collecting location data at city level rather than at an address level), controlling how data is stored (e.g., aggregating data across users), and/or other methods such as differential privacy.

Therefore, although the present disclosure broadly covers use of personal information data to implement one or more various disclosed embodiments, the present disclosure also contemplates that the various embodiments can also be implemented without the need for accessing such personal information data. That is, the various embodiments of the present technology are not rendered inoperable due to the lack of all or a portion of such personal information data.

It is understood that any specific order or hierarchy of blocks in the processes disclosed is an illustration of example approaches. Based upon design preferences, it is understood that the specific order or hierarchy of blocks in the processes may be rearranged, or that all illustrated blocks be performed. Any of the blocks may be performed simultaneously. In one or more implementations, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

As used in this specification and any claims of this application, the terms “base station”, “receiver”, “computer”, “server”, “processor”, and “memory” all refer to electronic or other technological devices. These terms exclude people or groups of people. For the purposes of the specification, the terms “display” or “displaying” means displaying on an electronic device.

As used herein, the phrase “at least one of” preceding a series of items, with the term “and” or “or” to separate any of the items, modifies the list as a whole, rather than each member of the list (i.e., each item). The phrase “at least one of” does not require selection of at least one of each item listed; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

The predicate words “configured to”, “operable to”, and “programmed to” do not imply any particular tangible or intangible modification of a subject, but, rather, are intended to be used interchangeably. In one or more implementations, a processor configured to monitor and control an operation or a component may also mean the processor being programmed to monitor and control the operation or the processor being operable to monitor and control the operation. Likewise, a processor configured to execute code can be construed as a processor programmed to execute code or operable to execute code.

Phrases such as an aspect, the aspect, another aspect, some aspects, one or more aspects, an implementation, the implementation, another implementation, some implementations, one or more implementations, an embodiment, the embodiment, another embodiment, some implementations, one or more implementations, a configuration, the configuration, another configuration, some configurations, one or more configurations, the subject technology, the disclosure, the present disclosure, other variations thereof and alike are for convenience and do not imply that a disclosure relating to such phrase(s) is essential to the subject technology or that such disclosure applies to all configurations of the subject technology. A disclosure relating to such phrase(s) may apply to all configurations, or one or more configurations. A disclosure relating to such phrase(s) may provide one or more examples. A phrase such as an aspect or some aspects may refer to one or more aspects and vice versa, and this applies similarly to other foregoing phrases.

The word “exemplary” is used herein to mean “serving as an example, instance, or illustration”. Any embodiment described herein as “exemplary” or as an “example” is not necessarily to be construed as preferred or advantageous over other implementations. Furthermore, to the extent that the term “include”, “have”, or the like is used in the description or the claims, such term is intended to be inclusive in a manner similar to the term “comprise” as “comprise” is interpreted when employed as a transitional word in a claim.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112(f) unless the element is expressly recited using the phrase “means for” or, in the case of a method claim, the element is recited using the phrase “step for”.

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein, but are to be accorded the full scope consistent with the language claims, wherein reference to an element in the singular is not intended to mean “one and only one” unless specifically so stated, but rather “one or more”. Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the subject disclosure.

What is claimed is:

1. A method comprising:
receiving an accelerometer signal from an accelerometer in a headphone configured to be mounted in a user's ear canal;
filtering the accelerometer signal to extract a cardiac signal;
detecting a plurality of peaks in the cardiac signal; and
determining a cardiac rate of the user based on the detected plurality of peaks.

2. The method of claim 1, wherein the accelerometer signal is parsed into an audio format.

3. The method of claim 2, wherein the audio format is a linear pulse code modulated format.

4. The method of claim 1, wherein filtering the accelerometer signal comprises applying a bandpass filter to the accelerometer signal.

5. The method of claim 4, wherein the bandpass filter is a finite impulse response Butterworth bandpass filter.

6. The method of claim 1, wherein detecting the plurality of peaks in the cardiac signal comprises performing template matching on the cardiac signal.

7. The method of claim 1, further comprising:
extracting an envelope of the cardiac signal,
wherein the plurality of peaks are detected in the extracted envelope of the cardiac signal.

8. A non-transitory computer-readable medium storing instructions which, when executed by one or more processors, cause the one or more processors to perform operations comprising:
receiving an accelerometer signal from an accelerometer in a headphone configured to be mounted in a user's ear canal;
filtering the accelerometer signal to extract a first cardiac signal and a second cardiac signal;
detecting a first plurality of peaks in the first cardiac signal and a second plurality of peaks in the second cardiac signal; and
determining a first cardiac rate of the user based on the detected first plurality of peaks and a second cardiac rate of the user based on the detected second plurality of peaks.

9. The non-transitory computer-readable medium of claim 8, wherein the accelerometer signal is parsed into an audio format.

10. The non-transitory computer-readable medium claim 9, wherein the audio format is a linear pulse code modulated format.

11. The non-transitory computer-readable medium claim 8, wherein filtering the accelerometer signal comprises applying a first bandpass filter to the accelerometer signal to extract the first cardiac signal and a second bandpass filter to the accelerometer signal to extract the second cardiac signal.

12. The non-transitory computer-readable medium of claim 8, wherein detecting the plurality of peaks in the cardiac signal comprises performing template matching on the first cardiac signal.

13. The non-transitory computer-readable medium of claim 8, further comprising:
extracting an envelope of the second cardiac signal,
wherein the plurality of peaks are detected in the extracted envelope of the second cardiac signal.

14. An electronic device, comprising:
a memory storing one or more computer programs; and
one or more processors configured to execute instructions of the one or more computer programs to:
receive an accelerometer signal from an accelerometer in a headphone configured to be mounted in a user's ear canal;
filter the accelerometer signal to extract a cardiopulmonary signal;
detect a plurality of peaks in the cardiopulmonary signal; and
determine a cardiopulmonary rate of the user based on the detected plurality of peaks.

15. The electronic device of claim 14, wherein the accelerometer signal is parsed into an audio format.

16. The electronic device of claim 14, wherein filtering the accelerometer signal comprises applying a bandpass filter to the accelerometer signal.

17. The electronic device of claim 14, wherein the cardiopulmonary rate of the user is a respiratory rate of the user.

18. The electronic device of claim 14, wherein the headphone is positioned on the chest of the user.

19. A headphone, comprising:
an accelerometer;
a communication module;
a memory storing one or more computer programs; and
a processor configured to execute instructions in the one or more computer programs to:
capture an accelerometer signal from the accelerometer;
parse the accelerometer signal into an audio format; and
transmit the accelerometer signal in the audio format to an electronic device using the communication module.

20. The headphone of claim 19, wherein the audio format is a linear pulse code modulated format.

21. The headphone of claim 19, wherein the communication module is a wireless communication module.
